(19) United States
(12) Patent Application Publication
(10) Pub. No.: US 2015/0121997 A1
(43) Pub. Date: May 7, 2015

(54) NON-DESTRUCTIVE MEASUREMENT OF MECHANICAL PROPERTIES OF AN ELLIPSOIDAL SHELL

(71) Applicants: David Chuen Chun Lam, Hong Kong (CN); Wai Lun Ko, Hong Kong (CN); Ka Kit Leung, Hong Kong (CN)

(72) Inventors: David Chuen Chun Lam, Hong Kong (CN); Wai Lun Ko, Hong Kong (CN); Ka Kit Leung, Hong Kong (CN)

(73) Assignee: THE HONG KONG UNIVERSITY OF SCIENCE AND TECHNOLOGY, Hong Kong (CN)

(21) Appl. No.: 14/117,621

(22) PCT Filed: May 15, 2012
(86) PCT No.: PCT/CN12/00652

§ 371 Date: Nov. 13, 2013

Related U.S. Application Data
(60) Provisional application No. 61/457,784, filed on Jun. 3, 2011.

(51) Int. Cl. G01N 3/30; A61B 3/16

(52) U.S. Cl. CPC G01N 3/30; A61B 3/16
USPC 73/82; 600/405

(57) ABSTRACT

Systems and methods that facilitate the determination of mechanical properties of an ellipsoidal shell are provided in this disclosure. The ellipsoidal shell is contacted with an indenter device. The indenter device indents the ellipsoidal shell and creates an indentation in an indentation region on the ellipsoidal shell. Indentation data is recorded at the indentation region. Mechanical properties of the ellipsoid shell can be determined based on the indentation data.

### CROSS REFERENCE TO RELATED APPLICATIONS

This application claims the benefit of provisional application No. 61/457,784, entitled: “NON-INVASIVE METHODOLOGY TO MEASURE THE MECHANICAL PROPERTIES OF HOLLOW ELLIPSOIDS AND APPLICATIONS IN CORNEA” and filed on Jun. 3, 2011.

### TECHNICAL FIELD

This disclosure generally relates to measurement of mechanical properties of an ellipsoidal shell in a non-destructive manner.

### BACKGROUND

Mechanical properties of a material in a structure include stiffness and modulus. In non-biological applications, the mechanical properties of the material in the structure are important factors in the design and selection of materials for the structure, in operation of the structure, and in failure analysis of the structure. In biological applications, the mechanical properties of biological tissue are used in illness diagnosis and in treatment monitoring.

When the structure is flat, the mechanical properties can be measured non-destructively using indentation methods or ultrasonic methods or destructively using a strip tensile test. When the structure is not flat, the mechanical properties can be measured using destructive method. An example of a destructive method is an inflation test. With an inflation test, a needle is inserted into the structure to control the interior pressure and the inflation is measured as a function of inflation pressure. The inflation is invasive, leaving a hole in the structure.

The above-described background is merely intended to provide an overview of contextual information surrounding measurement of mechanical properties of materials, and is not intended to be exhaustive. Additional context may become apparent upon review of one or more of the various non-limiting embodiments of the following detailed description.

### SUMMARY

The following presents a simplified summary of the specification in order to provide a basic understanding of some aspects of the specification. This summary is not an extensive overview of the specification. It is intended to neither identify key or critical elements of the specification nor delineate any scope of particular embodiments of the specification, or any scope of the claims. Its sole purpose is to present some concepts of the specification in a simplified form as a prelude to the more detailed description that is presented later.

In accordance with one or more embodiments and corresponding disclosure, various non-limiting aspects are described in connection with measuring mechanical properties of a material of a structure that is not flat in a non-destructive manner. In other words, the mechanical properties, such as stiffness and modulus, can be measured without destroying or otherwise damaging the material or the structure.

An example of a structure that is not flat is an ellipsoidal shell. In accordance with a non-limiting embodiment, stiffness and modulus of the ellipsoidal shell can be determined in a non-destructive manner. The ellipsoidal shell is contacted with an indenter device. The indenter device indents the ellipsoidal shell and creates an indentation region. Indentation data is recorded at the indentation region. Mechanical properties of the ellipsoid shell are determined based on the indentation data.

In a non-limiting embodiment, a method is described for determining a mechanical property of an ellipsoidal shell. The ellipsoidal shell is contacted by an indenter device, which indents the ellipsoidal shell at an indentation region. Indentation data is acquired from the indentation region. A mechanical property of the ellipsoidal shell is determined based on the indentation data.

In another non-limiting embodiment, a device is described that facilitates determination of a mechanical property of an ellipsoidal shell. The device includes an indenter and a calculator. The indenter at least partially contacts the ellipsoidal shell and creates an indentation region in the ellipsoidal shell in response to being subjected to a load. The calculator receives data about the load and associated displacement data for the indentation region, to determine a slope of the load data versus the displacement data, and to determine a mechanical property of the ellipsoidal shell based on the slope.

In a further non-limiting embodiment, a system is described to determine a mechanical property of an ellipsoidal shell. The system includes an indenter that contacts and applies a load to an ocular tissue and causes an indentation region to be formed in the ocular tissue. The system also includes a calculator that receives data about the load and associated displacement data for the indentation region, determines a slope of the load data versus the displacement data, and determines a mechanical property of the ellipsoidal shell based on the slope.

In another non-limiting embodiment, a system is described to facilitate determination of a mechanical property of an ellipsoidal shell. The system includes means for contacting and indenting an ellipsoidal shell resulting in an indentation region. The system also includes means for determining a mechanical property of the ellipsoidal shell based on indentation data measured from the indentation region.

The following description and the drawings set forth certain illustrative aspects of the specification. These aspects are indicative, however, of but a few of the various ways in which the various embodiments of the specification may be employed. Other aspects of the specification will become apparent from the following detailed description of the specification when considered in conjunction with the drawings.

### BRIEF DESCRIPTION OF THE DRAWINGS

Numerous aspects and embodiments are set forth in the following detailed description, taken in conjunction with the accompanying drawings, in which like reference characters refer to like parts throughout, and in which:

FIG. 1 illustrates an example non-limiting system that indents an ellipsoidal shell in a non-destructive manner, according to an embodiment of the disclosure;

FIG. 2 illustrates an example non-limiting system that facilitates measurement of mechanical properties of an ellipsoidal shell in a non-destructive manner, according to an embodiment of the disclosure;

FIG. 3 illustrates an example non-limiting system that facilitates diagnosis of a disease state of an ellipsoidal shell based on a mechanical property, according to an embodiment of the disclosure;

FIG. 4 illustrates an example non-limiting experimental setup for measuring mechanical properties of an ellipsoidal shell, according to an embodiment of the disclosure;

FIG. 5 illustrates graphs of load versus displacement for (A) a porcine cornea and (B) an artificial silicone rubber ellipsoid, according to an embodiment of the disclosure;

FIG. 6 illustrates a schematic diagram of the load-displacement slope extraction position, according to an embodiment of the disclosure;

FIG. 7 illustrates graphs of load-displacement data for different indentation rates of (A) a porcine cornea and (B) a silicone rubber ellipsoid, according to an embodiment of the disclosure;

FIG. 8 illustrates a typical relationship between a porcine corneal elastic modulus and the indentation rate, according to an embodiment of the disclosure;

FIG. 9 illustrates an example non-limiting method for measuring mechanical properties of an ellipsoidal shell in a non-destructive manner, according to an embodiment of the disclosure;

FIG. 10 illustrates an example non-limiting method for determining stiffness and modulus for an ellipsoidal shell, according to an embodiment of the disclosure;

FIG. 11 illustrates an example computing environment in which the various embodiments described herein can be implemented; and

FIG. 12 illustrates an example of a computer network in which various embodiments described herein can be implemented.

### DETAILED DESCRIPTION

Various aspects or features of this disclosure are described with reference to the drawings, wherein like reference numerals are used to refer to like elements throughout. In this specification, numerous specific details are set forth in order to provide a thorough understanding of this disclosure. It should be understood, however, that the certain aspects of disclosure may be practiced without these specific details, or with other methods, components, materials, etc. In other instances, well-known structures and devices are shown in block diagram form to facilitate description and illustration of the innovation.

It is to be appreciated that in accordance with one or more embodiments described in this disclosure, mechanical properties of a material of a structure that is not flat can be measured in a non-destructive manner.

As used herein, the term “mechanical properties” generally refers to any quantitative property of a material. Examples of mechanical properties of a material include: strength, density, ductility, fatigue limit, modulus, toughness, hardness, softness, plasticity, Poisson's ratio, and the like. Stiffness, modulus, and Poisson's ratio are described herein, but it will be understood that the systems and methods described herein can apply to any mechanical property.

The phrase “a structure that is not flat” generally refers to a structure that does not have a generally linear shape. An example of a “flat” structure is a sheet. An example of a “structure that is not flat” is an ellipsoidal structure, such as an ellipsoidal shell. Structures with an ellipsoidal shell can include any ellipsoidal structure, such as ocular tissue (tissue of an eye, including sclera, cornea, or the like). Ellipsoidal shells are described herein, but it will be understood that the systems and methods described herein can apply to any structure that is not flat.

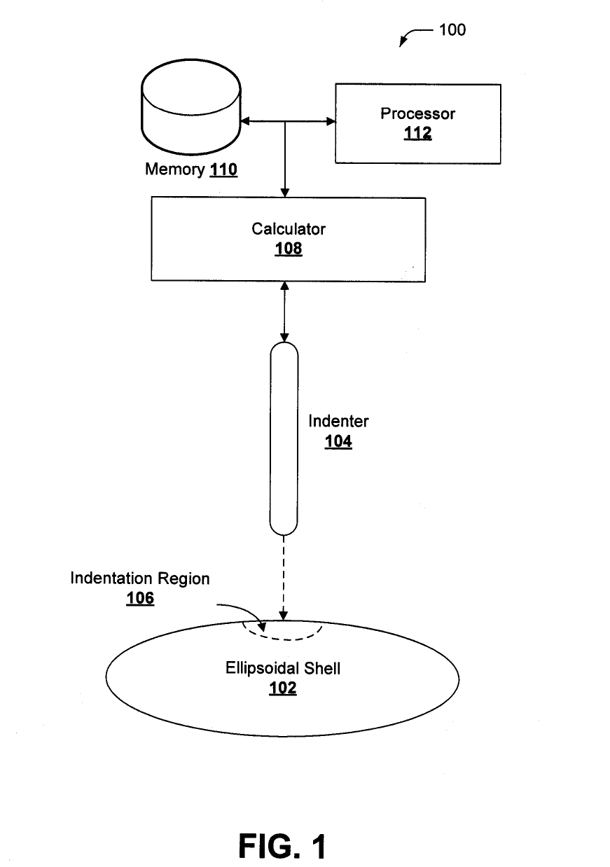

Referring now to the drawings, with reference initially to FIG. 1, a system 100 that indents an ellipsoidal shell in a non-destructive manner is set forth, according to an embodiment of the disclosure. The system 100 includes an indenter 104. An indenter 104 is any device that can at least partially contact an ellipsoidal shell 102 and create an indentation region 106 in the ellipsoidal shell 102 when subjected to a load. The indenter 104 can be any shape that can facilitate creation of the indentation region 106 in the ellipsoidal shell 102. In an embodiment, the indenter 104 is an axial-symmetric shape and creates an axial-symmetric indentation region 106. The indenter 104 can create the indentation region 106 in both a partial ellipsoidal shell 102 and a complete ellipsoidal shell 102.

The ellipsoidal shell 102, according to an embodiment, is a non-biological structure. In the case where the ellipsoidal shell 102 is non-biological material, the indenter can contact the ellipsoidal shell 102.

In another embodiment, the ellipsoidal shell 102 is a biological structure, such as ocular tissue. Examples of ocular tissue include sclera tissue or cornea tissue. In the case where the ellipsoidal shell 102 is biological tissue, the indenter can contact the ellipsoidal shell 102 in vivo or ex vivo.

The indenter 104 can be made of any material with a strength sufficient to withstand a load while creating the indentation region 106. In an embodiment, the indenter 104 is constructed at least partially of a material that is approved by the U.S. Food and Drug Administration as biocompatible. According to a further embodiment, the indenter 104 is of at least a material that is oxygen permeable. In another embodiment, the indenter 104 can initiate biofeedback treatment of the biological tissue based on a mechanical property.

The system 100 can also include a calculator 108 coupled to a memory 110 and a processor 112. In an embodiment, the memory 110, processor 112, and calculator 108 are part of a computing device.

According to an embodiment, the calculator 108 receives data about the indentation region 106 and determines a mechanical property of the ellipsoidal shell 102 based on the data about the indentation region 106. The data about the indentation region 106 can include load data and associated displacement data for the indentation region 106. Based on the load data and the associated displacement data, the calculator 108 can determine a slope of the load data versus the displacement data and determine a mechanical property of the ellipsoidal shell 102 based on the slope.

The mechanical property determined by the calculator 108, according to an embodiment, is stiffness. The stiffness is directly proportional to the slope of the load data versus the displacement data.

According to another embodiment, the mechanical property determined by the calculator 108 is modulus. The modulus can be an elastic modulus or a tangent modulus. The calculator 108 can determine the modulus based on the slope of the load data versus the displacement data and a geometric function. In an embodiment where a pressure inside the ellipsoidal shell is constant, the modulus can be determined according to:

$$E = \frac{{a}_{2} ( S , v , R , t ) \cdot ( R - t / 2 ) \sqrt{1 - {v}^{2}}}{{t}^{2}} \frac{d F}{d δ} ,$$

where E is the modulus of the ellipsoidal shell, F is the indentation load data, δ is the displacement data, and dF/dδ is the slope of the load data versus the displacement data. Additionally, a₂(S, ν, R, t) is a geometric function, where S is a shape of the indenter device, ν is Poisson's ratio, R is a radius of curvature of the ellipsoid shell, and t is the thickness of the ellipsoidal shell at the indentation region.

The following embodiment describes how system 100 can determine the stiffness and modulus of a hollow ellipsoidal shell from the load-displacement data in the indentation region 106. The indenter 104 can be axial-symmetric. The data analysis can be modified on the basis of the indentation relation for a hollow ellipsoid.

A formula for the displacement generated by the partial contact indentation on an ellipsoid with a flat punch is:

$$δ = a \frac{{FR}_{2} \sqrt{1 - {v}^{2}}}{{Et}^{2}} ,$$

where δ is the deflection (or displacement) under the center of the load, a is a geometric constant, F is the load concentrated on a small cir circular contact area by the flat punch, ν is the Poisson's ratio of the hollow ellipsoid, E is the elastic modulus of the material of the ellipsoid, t is the thickness of the hollow ellipsoid, R₂ is the radius of curvature of the hollow ellipsoid and can be presented as,

R₂=R−t/2

where R is the radius of curvature of the outermost surface of the hollow ellipsoid.

The parameter “a” can be determined based on μ, where μ is determined according to:

$$μ = {{r}_{o}^{′} [ \frac{12 ( 1 - {v}^{2} )}{{R}_{2}^{2} {t}^{2}} ]}^{1 / 4} ,$$

where r₀′ is the determined by:

$$\{ \begin{matrix}{r}_{o}^{′} = \sqrt{1.6 {r}_{0}^{2} + {t}^{2}} - 0.675 t , & if {r}_{o} < 0.5 t \\ {r}_{o}^{′} = {r}_{o} , & if {r}_{o} \geq 0.5 t\end{matrix} ,$$

where r₀ is the radius of the circular contact area by the partially contacted flat punch indenter.

The relationship between μ and a is shown below in Table 1:

| | μ | | | | | | | | |
|---|---|---|---|---|---|---|---|---|---|
| | 0 | 0.1 | 0.2 | 0.4 | 0.6 | 0.8 | 1.0 | 1.2 | 1.4 |
| a | 0.433 | 0.431 | 0.425 | 0.408 | 0.386 | 0.362 | 0.337 | 0.311 | 0.286 |

Rearranging

$$δ = a \frac{{FR}_{2} \sqrt{1 - {v}^{2}}}{{Et}^{2}}$$

can give an equation relating modulus E to the geometric parameters and the load-displacement data by partially contacted flat punch indentation:

$$E = \frac{a ( R - t / 2 ) \sqrt{1 - {v}^{2}}}{{t}^{2}} \frac{F}{δ} ,$$

where

$$\frac{F}{δ}$$

is the slope of the load-displacement data from the partially contacted flat punch indentation, and is defined as the stiffness of the hollow ellipsoid.

The equation,

$$E = \frac{a ( R - t / 2 ) \sqrt{1 - {v}^{2}}}{{t}^{2}} \frac{F}{δ} ,$$

can be further generalized so that the equation can be applicable to the indentation of ellipsoids by any partially contacted axial-symmetric indenter:

$$E = \frac{{a}_{2} ( S , v , R , t ) \cdot ( R - t / 2 ) \sqrt{1 - {v}^{2}}}{{t}^{2}} \frac{F}{δ} ,$$

where a₂(S,ν,R,t) is a geometric function governed by the shape of the partially contacted axial-symmetric indenter and both the Poisson's ratio and the geometrical parameters of the ellipsoid.

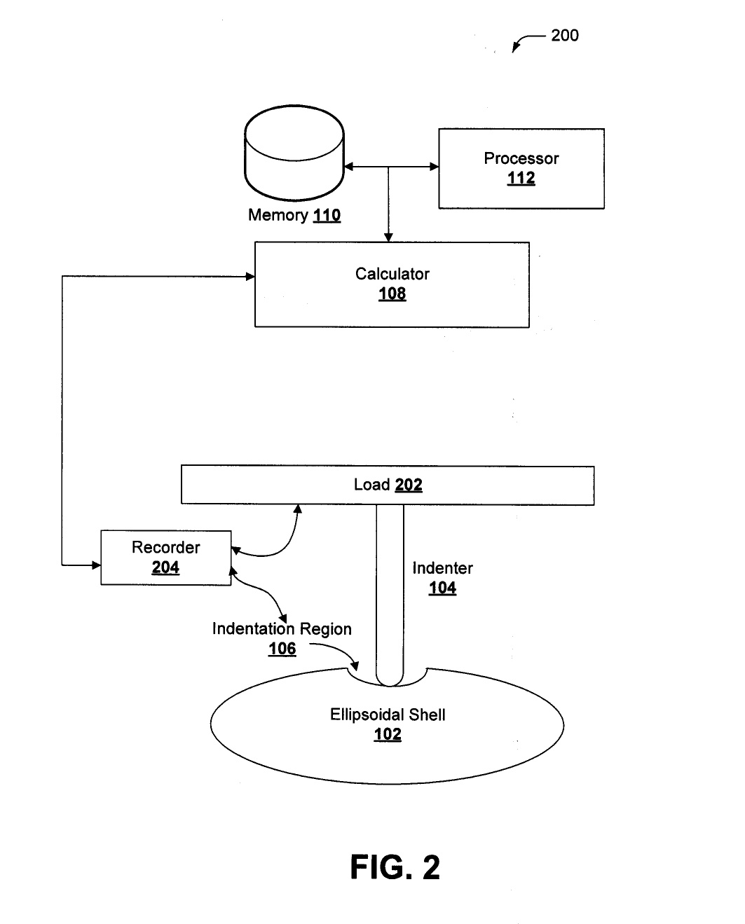

Referring now to FIG. 2, illustrated is a system 200 that facilitates measurement of mechanical properties of an ellipsoidal shell 102 in a non-destructive manner, according to an embodiment of the disclosure. FIG. 2 illustrates a case where the indenter 104 creates the indentation region 106 in the ellipsoidal shell through compression by a load 202. The system includes a recorder 204 that records data related to the indentation region 106 (e.g., load data and displacement data). Although illustrated as a separate component, the recorder 204 can also be part of the indenter 104, the load 202, the calculator 108 or any other component. According to an embodiment, the recorder 204 is constructed of one or more sensors or transducers. For example, the recorder 202 can be constructed of a sensor that senses the displacement and sensor that senses the load; data from the sensors can be provided to the calculator 108. The calculator 108 uses the data provided by the recorder 204 to determine the mechanical property.

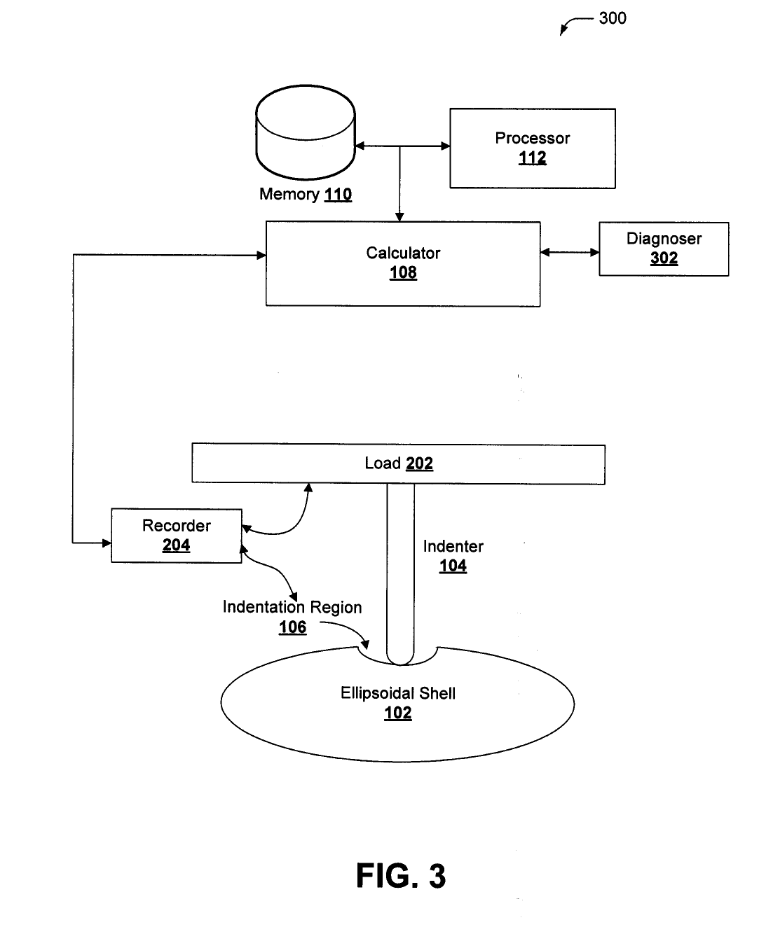

Referring now to FIG. 3, illustrated is a system 300 that facilitates diagnosis of a disease state of an ellipsoidal shell 102 based on a mechanical property, according to an embodiment of the disclosure. In the embodiment of FIG. 3, the elliptical shell 102 is a biological structure. The biological structure, in an embodiment, includes ocular tissue. The ocular tissue can include sclera tissue or cornea tissue.

The system 300 includes a diagnoser 302. Although shown as a separate component, the diagnoser 302 can be a part of the calculator 108. The diagnoser 302 can also be included in a computing device with the calculator 108, memory 110 and processor 112.

The diagnoser 302 can facilitate a medical diagnosis based on the mechanical property. In an embodiment, the diagnoser 302 can produce a suggested diagnosis based on the mechanical property (e.g., flagging data in an output indicating a disease state). According to another embodiment, the diagnoser 302 can diagnose a disease state based on the mechanical property and trigger a biofeedback or treatment procedure. For example, the diagnoser 302 diagnoses a disease state, the diagnoser 302 can trigger administration of a treatment modality through a component of system 300, such as indenter 104.

The diagnoser 302 can include a database listing disease parameters for the biological tissue with respect to the mechanical property. For example, if the biological tissue is ocular tissue, the database can list mechanical properties for glaucoma. In glaucoma, mechanical properties of stiffness or modulus can be altered from normal. The database in the diagnoser 302 can include threshold values for stiffness or modulus where values outside of the threshold indicate a diagnosis of glaucoma. The diagnoser 302 can facilitate diagnosis, risk assessment and treatment for monitoring of illness in the biological tissue (such as optic illness).

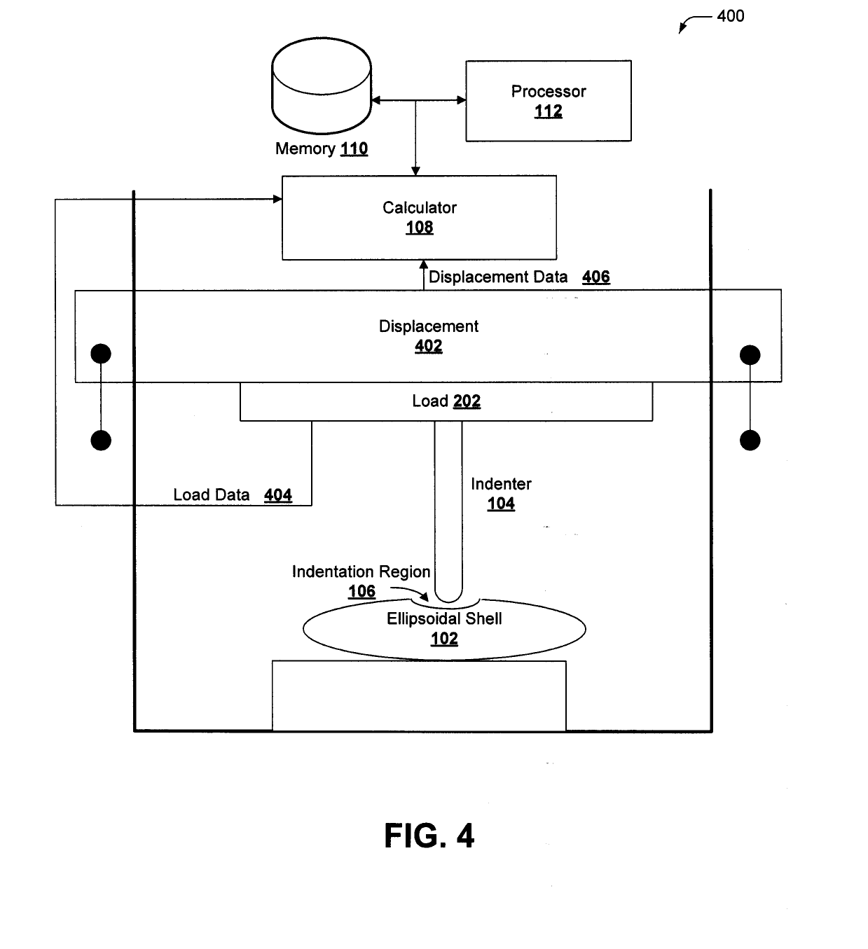

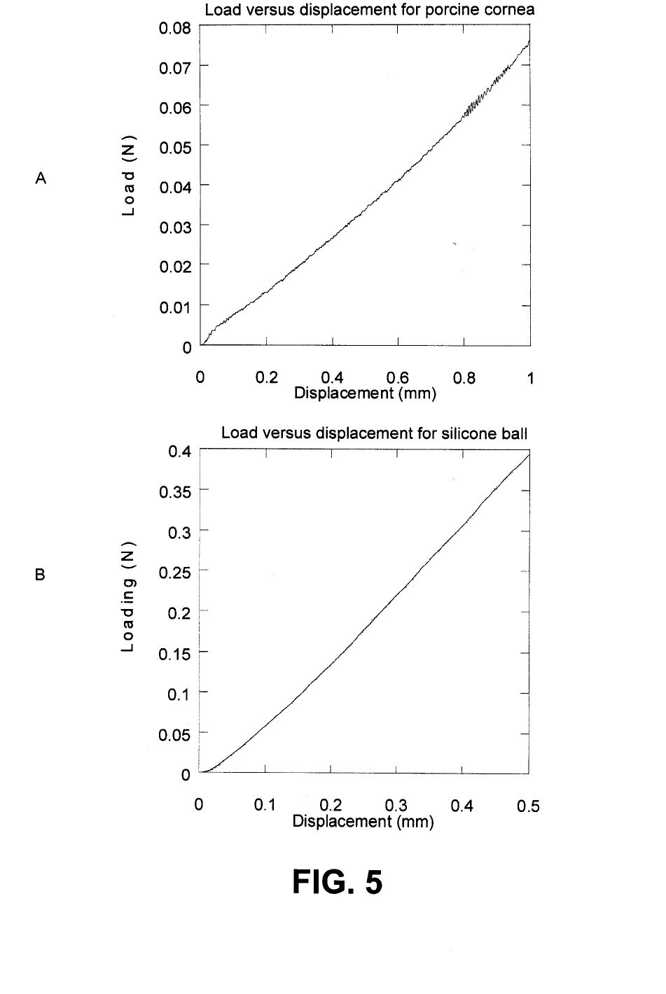

FIGS. 5-8 illustrate an experiment and associated outcomes, proving the efficacy of the non-invasive test for mechanical properties described herein. FIG. 4 generally illustrates an experimental setup for measurement of mechanical properties of an ellipsoidal shell (porcine cornea or artificial silicone rubber ellipsoid). FIGS. 5-8 show relationships between data recorded with the experimental setup and the mechanical properties of the ellipsoidal shell.

Referring now to FIG. 4, illustrated is an example experimental setup for measuring mechanical properties of an ellipsoidal shell 102, according to an embodiment of the disclosure. The ellipsoidal shell 102 can be a biological structure (a porcine cornea) or a non-biological structure (artificial silicone rubber ellipsoid).

The experimental setup can include a displacement detector 402 and a load 202 with displacement data 406 and load data 404 transmitted to calculator 108. The experimental setup can include a universal testing machine (MTS, Alliance RT/5) and a 10N load cell.

The experimental setup can be used to demonstrate the measurement of stiffness and modulus in a non-destructive manner. The stiffness and modulus can be determined when the indenter 104 partially contacts the elliptical shell 102. The slope of the load-displacement curve can be extracted to achieve a measure of the stiffness. The slope,

$$\frac{F}{δ} ,$$

of the load-displacement curve can also be used to calculate the modulus according to:

$$E = \frac{{a}_{2} ( S , v , R , t ) \cdot ( R - t / 2 ) \sqrt{1 - {v}^{2}}}{{t}^{2}} \frac{F}{δ} ,$$

where a₂(S,ν,R,t) is a geometric function governed by the shape of the indenter 104 and both the Poisson's ratio and the geometrical parameters of the ellipsoidal shell 102.

Referring now to FIG. 5, graphs of load versus displacement for (A) a porcine cornea and (B) an artificial silicone rubber ellipsoid are shown, according to an embodiment of the disclosure. In both cases, the load versus displacement plots are substantially linear. The porcine cornea exhibits more variability than the silicone rubber ellipsoid. However, the porcine cornea exhibits a linear region of the graph for which a slope can be calculated.

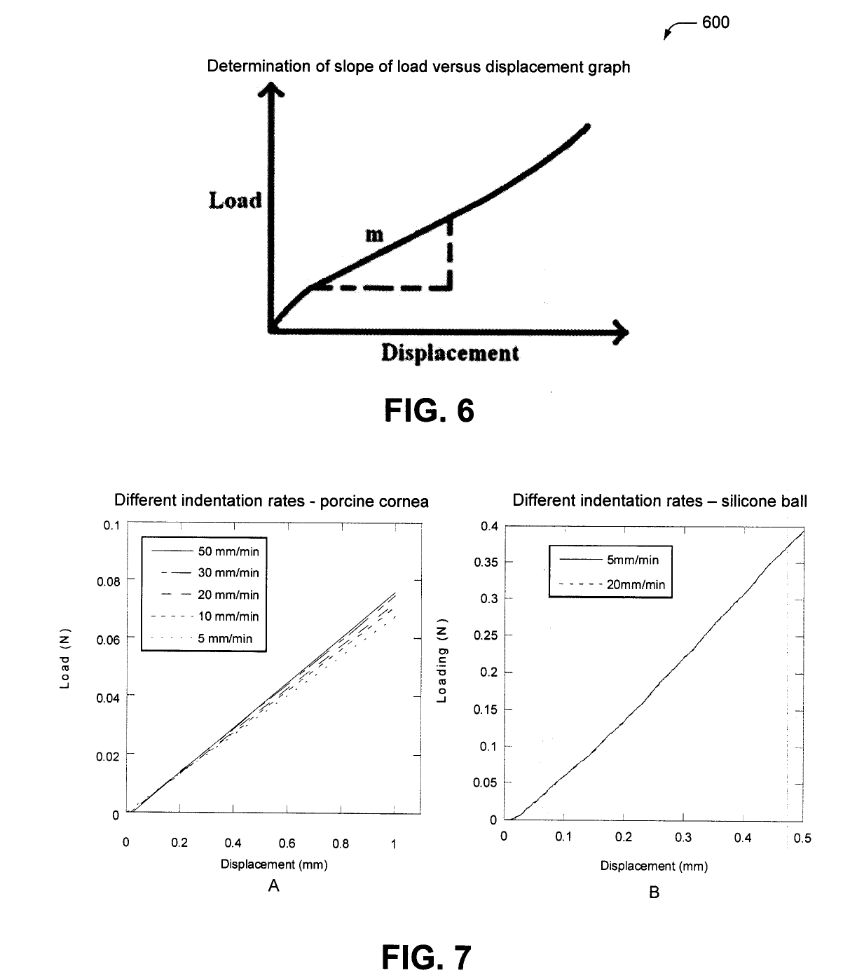

FIG. 6 illustrates a schematic diagram of the load-displacement slope extraction position, according to an embodiment of the disclosure. The slope is extracted from a region of the load vs. displacement curve that is substantially linear. Another option is to take the slope of a linear regression of a portion of the load vs. displacement curve.

After determining the slope, which corresponds to stiffness, FIG. 7 shows graphs of stiffness versus indentation rates for (A) a porcine cornea and (B) a silicone rubber ellipsoid, according to an embodiment of the disclosure. For both the porcine cornea and the silicone rubber ellipsoid, for different indentation rates, the slope was a good predictor of stiffness. The porcine cornea exhibited more variability than the silicone rubber ellipsoid, but after a rate threshold, the slope is reproducible. According to an embodiment, the rate threshold is 20 mm/min.

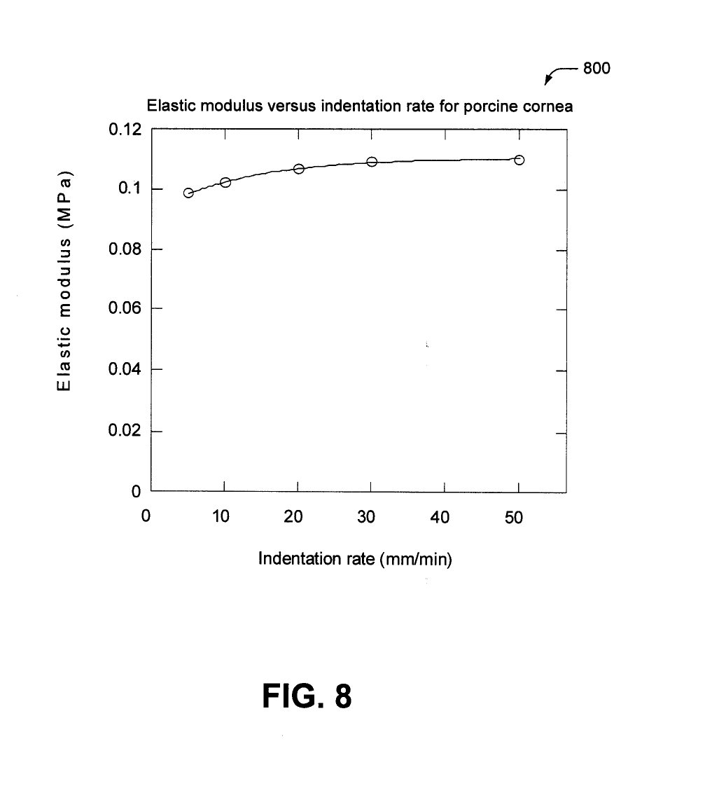

The elastic modulus also depends on indentation rate. FIG. 8 shows a typical relationship between a porcine corneal elastic modulus and the indentation rate, according to an embodiment of the disclosure.

The elastic modulus of the porcine cornea depends on indentation rate. However, elastic modulus obtained above a rate threshold is reproducible. At an indentation rate of 20 mm/min, the average corneal stiffness and elastic modulus of the porcine cornea were determined to be 0.068±0.007 N/mm and 0.14±0.04 MPa (n=12) respectively. This elastic modulus is in good agreement with the values found by other measurement methods.

The stiffness and the elastic modulus of a non-biological silicon rubber ellipsoid do not depend on the indentation rate. The stiffness was found to be 0.821 N/mm and the elastic modulus was found to be 1.56 MPa. The elastic modulus of the silicon rubber ellipsoid of 1.56 MPa is in good agreement with the value found by the standard 3-point bending tests performed on a silicone rubber plate with the same composition of materials of 1.55 MPa.

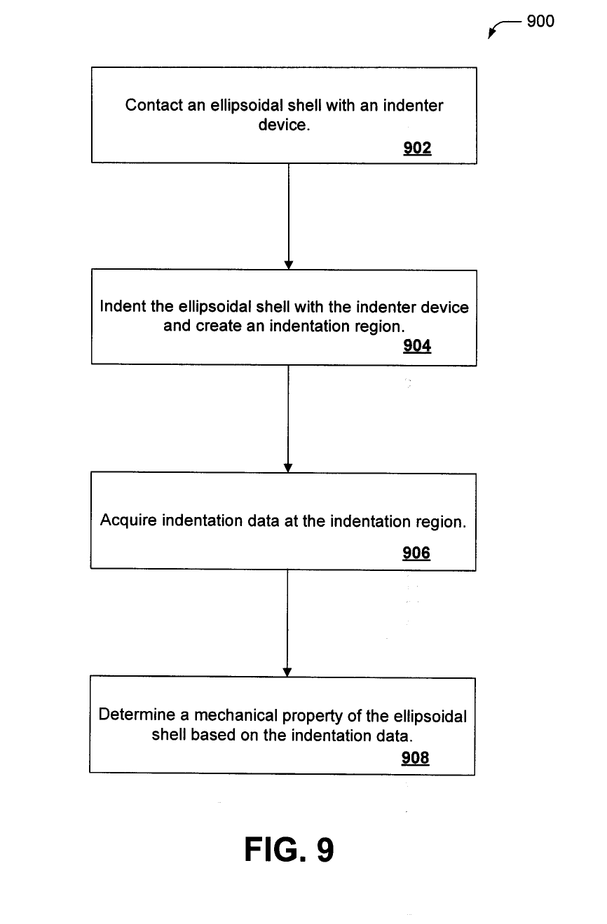

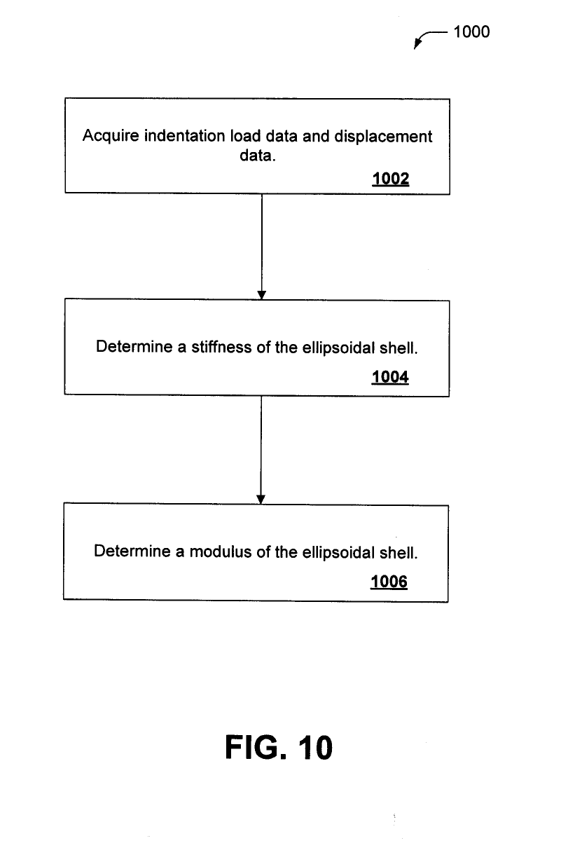

FIGS. 9 and 10 illustrate methods and/or flow diagrams in accordance with embodiments of this disclosure. For simplicity of explanation, the methods are depicted and described as a series of acts. However, acts in accordance with this disclosure can occur in various orders and/or concurrently, and with other acts not presented and described in this disclosure. Furthermore, not all illustrated acts may be required to implement the methods in accordance with the disclosed subject matter. In addition, those skilled in the art will understand and appreciate that the methods could alternatively be represented as a series of interrelated states via a state diagram or events. Additionally, it should be further appreciated that the methods disclosed in this specification are capable of being stored on an article of manufacture to facilitate transporting and transferring such methods to computing devices. The term article of manufacture, as used in this disclosure, is intended to encompass a computer program accessible from any computer-readable device or computer-readable storage/communications media.

Referring to FIG. 9, presented is a flow diagram of a method 900 for measuring mechanical properties of an ellipsoidal shell in a non-destructive manner, according to an embodiment of the disclosure. At element 902, an ellipsoidal shell is contacted with an indenter device. The ellipsoidal shell can be an intact ellipsoidal shell or a partial ellipsoidal shell. The ellipsoidal shell can be made of a biological material or a non-biological material. In an embodiment, the biological tissue is ocular tissue. The ocular tissue can be sclera, cornea, or any other type of ocular tissue.

At 904, the ellipsoidal shell is indented with the indenter device to create an indentation region. At 906, indentation data is acquired at the indentation region. The indentation data can include indentation load data and associated displacement data. At 908, a mechanical property of the ellipsoidal shell is determined based on the indentation data. The mechanical property, according to an embodiment, can be stiffness or modulus. The modulus can be a tangent modulus, an elastic modulus, or the like.

Referring to FIG. 10, presented above is a flow diagram of a method 1000 for determining stiffness and modulus for an ellipsoidal shell, according to an embodiment of the disclosure. At 1002, indentation load data and displacement data are acquired. At 1004, a stiffness of the ellipsoidal shell is determined. In an embodiment, the stiffness is determined based on a slope of the load data versus the displacement data. At 1006, a modulus of the ellipsoidal shell is determined. The modulus is determined when a pressure inside the ellipsoidal shell is constant, according to:

$$E = \frac{{a}_{2} ( S , v , R , t ) \cdot ( R - t / 2 ) \sqrt{1 - {v}^{2}}}{{t}^{2}} \frac{d F}{d δ} ,$$

E is the modulus of the ellipsoidal shell. F is the indentation load data, δ is the displacement data, and dF/dδ is the slope of the load data versus the displacement data. Additionally, a₂(S, ν, R, t) is a geometric function, where S is a shape of the indenter device, ν is Poisson's ratio, R is a radius of curvature of the ellipsoid shell, and t is the thickness of the ellipsoidal shell at the indentation region.

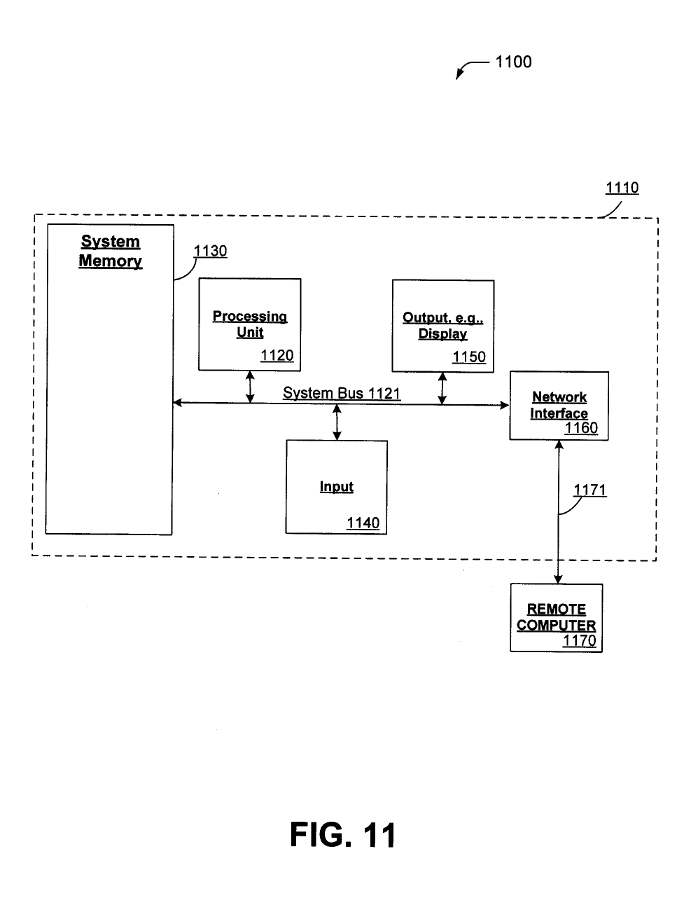

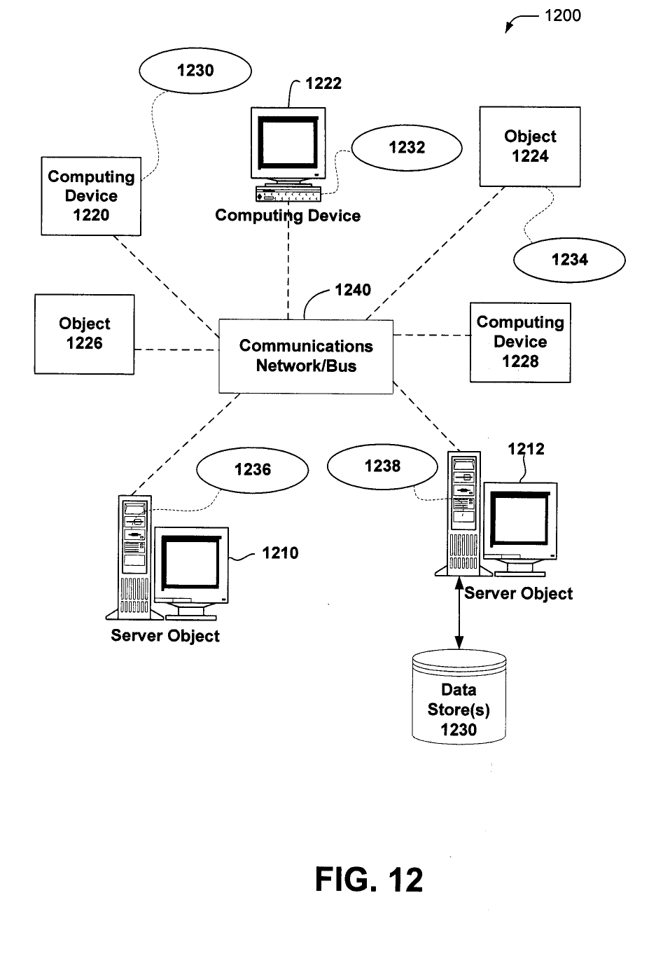

The systems and methods (e.g., calculator 108) described above can be implemented in software, hardware, or a combination thereof. FIGS. 11 and 12 provide hardware context for the devices, user interfaces and methods described above. FIG. 11 illustrates a computing environment 1100 that can be utilized in connection with the devices, user interfaces and methods described above. FIG. 12 illustrates a computing network 1200 that can be utilized in connection with facilitating the systems and methods described above. It should be appreciated that artificial intelligence can also be utilized to implement the systems and methods described herein.

Referring now to FIG. 11, illustrated is an example of a suitable computing system environment 1100 in which one or more of the embodiments can be implemented. The computing system environment 1100 is just one example of a suitable computing environment and is not intended to suggest any limitation as to the scope of use or functionality of any of the embodiments. Neither should the computing environment 1100 be interpreted as having any dependency or requirement relating to any one or combination of components illustrated in the exemplary operating environment 1100.

With reference to FIG. 11, the computing system environment 1100 can include computer 1110, which can be a handheld or non-handheld computer. The computer 1110 need only be capable of interfacing with a testing device (e.g., a device that records indentation data). However, the computing system environment 1100 can be any other computing device with a processor to execute the methods described herein and a display, such as a desktop computer, a laptop computer, a mobile phone, a mobile internet device, a tablet, or the like. Components of the computer 1110 can include, but are not limited to, a processing unit 1120, a system memory 1130, and a system bus 1121 that couples various system components including the system memory to the processing unit 1120. For example, the methods described herein can be stored in the system memory 1130 and executed by the processing unit 1120.

The computer 1110 can also include a variety of computer readable media, and can be any available media that can be accessed by computer 1110. The system memory 1130 can include computer storage media in the form of volatile and/or nonvolatile memory such as read only memory (ROM) and/or random access memory (RAM). By way of example, and not limitation, memory 1130 can also include an operating system, application programs, other program modules, and program data.

Computing devices typically include a variety of media, which can include computer-readable storage media and/or communications media, which two terms are used herein differently from one another as follows.

Computer-readable storage media can be any available storage media that can be accessed by the computer and includes both volatile and nonvolatile media, removable and non-removable media. By way of example, and not limitation, computer-readable storage media can be implemented in connection with any method or technology for storage of information such as computer-readable instructions, program modules, structured data, or unstructured data. Computer-readable storage media can include, but are not limited to, RAM, ROM, EEPROM, flash memory or other memory technology, CD-ROM, digital versatile disk (DVD) or other optical disk storage, magnetic cassettes, magnetic tape, magnetic disk storage or other magnetic storage devices, or other tangible and/or non-transitory media which can be used to store desired information. Computer-readable storage media can be accessed by one or more local or remote computing devices, e.g., via access requests, queries or other data retrieval protocols, for a variety of operations with respect to the information stored by the medium.

Communications media typically embody computer-readable instructions, data structures, program modules or other structured or unstructured data in a data signal such as a modulated data signal, e.g., a carrier wave or other transport mechanism, and includes any information delivery or transport media. The term “modulated data signal” or signals refers to a signal that has one or more of its characteristics set or changed, in such a manner as to encode information in one or more signals. By way of example, and not limitation, communication media include wired media, such as a wired network or direct-wired connection, and wireless media such as acoustic, RF, infrared and other wireless media.

A user can enter commands and information into the computer 1110 through input devices 1140, such as entering the indentation data. A monitor or other type of display device, e.g., touch screen or virtual display, can also connect to the system bus 1121 via an interface, such as output interface 1150.

The computer 1110 can operate in a networked or distributed environment using logical connections to one or more other remote computers, such as remote computer 1170. The remote computer 1170 can be a personal computer, a server, a router, a network PC, a peer device or other common network node, or any other remote media consumption or transmission device, and can include any or all of the elements described above relative to the computer 1110. The logical connections depicted in FIG. 11 include a network 1171, such local area network (LAN) or a wide area network (WAN), but can also include other networks/buses. Such networking environments are commonplace in homes, offices, enterprise-wide computer networks, intranets and the Internet.

Referring now to FIG. 12, illustrated is a schematic diagram of an exemplary networked or distributed computing environment 1200. The computer 1110 of FIG. 11 can be operational in the network of FIG. 12. The distributed computing environment comprises computing objects 1210, 1212, etc. and computing objects or devices 1220, 1222, 1224, 1226, 1228, etc., which can include programs, methods, data stores, programmable logic, etc., as represented by applications 1230, 1232, 1234, 1236, 1238. It can be appreciated that objects 1210, 1212, etc. and computing objects or devices 1220, 1222, 1224, 1226, 1228, etc. can comprise different devices, such as remote controllers, PDAs, audio/video devices, mobile phones, MP3 players, laptops, etc.

Each object 1210, 1212, etc. and computing objects or devices 1220, 1222, 1224, 1226, 1228, etc. can communicate with one or more other objects 1210, 1212, etc. and computing objects or devices 1220, 1222, 1224, 1226, 1228, etc. by way of the communications network 1240, either directly or indirectly. Even though illustrated as a single element in FIG. 12, network 1240 can comprise other computing objects and computing devices that provide services to the system of FIG. 12, and/or can represent multiple interconnected networks, which are not shown. Each object 1210, 1212, etc. or 1220, 1222, 1224, 1226, 1228, etc. can also contain an application, such as applications 1230, 1232, 1234, 1236, 1238, that might make use of an API, or other object, software, firmware and/or hardware, suitable for communication with various components relating to mechanical property measurement as provided in accordance with various embodiments.

There are a variety of systems, components, and network configurations that support distributed computing environments. For example, computing systems can be connected together by wired or wireless systems, by local networks or widely distributed networks. Currently, many networks are coupled to the Internet, which provides an infrastructure for widely distributed computing and encompasses many different networks, though any network infrastructure can be used for exemplary communications made incident to the techniques as described in various embodiments.

As a further non-limiting example, various embodiments described herein apply to any handheld, portable and other computing devices and computing objects of all kinds are contemplated for use in connection with the various embodiments described herein, i.e., anywhere that a device can request pointing based services. Accordingly, the general purpose remote computer described below in FIG. 12 is but one example, and the embodiments of the subject disclosure can be implemented with any client having network/bus interoperability and interaction.

Although not required, any of the embodiments can partly be implemented via an operating system, for use by a developer of services for a device or object, and/or included within application software that operates in connection with the operable component(s). Software can be described in the general context of computer executable instructions, such as program modules, being executed by one or more computers, such as client workstations, servers or other devices. Those skilled in the art will appreciate that network interactions can be practiced with a variety of computer system configurations and protocols.

What has been described above includes examples of the embodiments of the subject disclosure. It is, of course, not possible to describe every conceivable combination of components or methods for purposes of describing the claimed subject matter, but it is to be appreciated that many further combinations and permutations of this innovation are possible. Accordingly, the claimed subject matter is intended to embrace all such alterations, modifications, and variations that fall within the spirit and scope of the appended claims. Further, the order in which some or all of the process blocks appear in each process should not be deemed limiting. Rather, it should be understood that some of the process blocks can be executed in a variety of orders that are not illustrated in this disclosure. Moreover, the above description of illustrated embodiments of this disclosure, including what is described in the Abstract, is not intended to be exhaustive or to limit the disclosed embodiments to the precise forms disclosed. While specific embodiments and examples are described in this disclosure for illustrative purposes, various modifications are possible that are considered within the scope of such embodiments and examples, as those skilled in the relevant art can recognize.

In particular and in regard to the various functions performed by the above described components, modules, systems and the like, the terms used to describe such components are intended to correspond, unless otherwise indicated, to any component which performs the specified function of the described component (e.g., a functional equivalent), even though not structurally equivalent to the disclosed structure, which performs the function in the herein illustrated exemplary aspects of the claimed subject matter. The aforementioned systems, devices, and circuits have been described with respect to interaction between several components and/or blocks. It can be appreciated that such systems, devices, circuits, and components and/or blocks can include those components or specified sub-components, some of the specified components or sub-components, and/or additional components, and according to various permutations and combinations of the foregoing. Sub-components can also be implemented as components communicatively coupled to other components rather than included within parent components (hierarchical). Additionally, it should be noted that one or more components may be combined into a single component providing aggregate functionality or divided into several separate sub-components, and any one or more middle layers, such as a management layer, may be provided to communicatively couple to such sub-components in order to provide integrated functionality. Any components described in this disclosure may also interact with one or more other components not specifically described in this disclosure but known by those of skill in the art.

In addition, while a particular detail may have been disclosed with respect to only one of several embodiments, such feature may be combined with one or more other features of the other embodiments as may be desired and advantageous for any given or particular application. Furthermore, to the extent that the terms “includes,” “including,” “has,” “contains,” variants thereof, and other similar words are used in either the detailed description or the claims, these terms are intended to be inclusive in a manner similar to the term “comprising” as an open transition word without precluding any additional or other elements.

What is claimed is:

1. A method, comprising:
contacting an ellipsoidal shell by an indenter device;
indenting the ellipsoidal shell by the indenter device including creating an indentation region;
acquiring indentation data at the indentation region;
determining a mechanical property of the ellipsoidal shell based on the indentation data.

2. The method of claim 1, wherein the determining further comprises determining a stiffness of the ellipsoidal shell or a modulus of the ellipsoidal shell based on the indentation data.

3. The method of claim 1, wherein the acquiring further comprises acquiring indentation load data and associated displacement data in the indentation region.

4. The method of claim 3, wherein the determining further comprises determining a stiffness of the ellipsoidal shell based on a slope of the load data versus the displacement data.

5. The method of claim 4, wherein the determining further comprises determining a modulus of the ellipsoidal shell when a pressure inside the ellipsoidal shell is constant according to:
$$E = \frac{{a}_{2} ( S , v , R , t ) \cdot ( R - t / 2 ) \sqrt{1 - {v}^{2}}}{{t}^{2}} \frac{d F}{d δ} ,$$
where:
E is the modulus of the ellipsoidal shell,
F is the indentation load data,
δ is the displacement data,
dF/dδ is a slope of the load data versus the displacement data,
a₂(S, ν, R, t) is a function of geometric parameters, where:
S is a shape of the indenter device,
ν is Poisson's ratio,
R is a radius of curvature of the ellipsoidal shell, and
t is a thickness of the ellipsoidal shell at the indentation region.

6. The method of claim 2, wherein the determining the modulus further comprises determining a tangent modulus of the ellipsoidal shell or an elastic modulus of the ellipsoidal shell.

7. The method of claim 1, wherein the contacting further comprises contacting an intact ellipsoidal shell by the indenter device.

8. The method of claim 1, wherein the contacting further comprises contacting a partial ellipsoidal shell by the indenter device.

9. A device, comprising:
an indenter configured to at least partially contact an ellipsoidal shell and to create an indentation region in the ellipsoidal shell in response to being subjected to a load; and
a calculator configured to receive data about the load and associated displacement data for the indentation region, to determine a slope of the load data versus the displacement data, and to determine a mechanical property of the ellipsoidal shell based on the slope.

10. The device of claim 9, wherein the calculator determines a stiffness of the ellipsoidal shell based on the slope and a modulus of the ellipsoidal shell based on the stiffness and a function of a shape of the indenter, Poisson's ratio, a radius of curvature of the ellipsoidal shell, and a thickness of the ellipsoidal shell at the indentation region

11. The device of claim 9, wherein the ellipsoidal shell is non-biological tissue.

12. The device of claim 11, wherein the indenter is configured to contact the non-biological tissue ex vivo.

13. The device of claim 9, wherein the ellipsoidal shell is biological tissue.

14. The device of claim 13, wherein the biological tissue is ocular tissue.

15. The device of claim 14, wherein the ocular tissue is cornea tissue or sclera tissue.

16. The device of claim 13, wherein the indenter is configured to contact the biological tissue in vivo.

17. The device of claim 16, further comprising a diagnoser that facilitates a medical diagnosis based on the modulus of the biological tissue.

18. The device of claim 9, wherein the indenter is at least partially constructed of an oxygen-permeable material.

19. The device of claim 9, wherein the indenter is axial-symmetric.

20. The device of claim 9, wherein the indenter facilitates biofeedback treatment.

21. A system, comprising:
an indenter configured to contact and apply a load to an ocular tissue and to cause an indentation region to be formed in the ocular tissue; and
a calculator configured to receive data about the load and associated displacement data for the indentation region, determine a slope of the load data versus the displacement data, and determine a mechanical property of the ellipsoidal shell based on the slope.

22. The system of claim 21, wherein indenter is configured to contact the ocular tissue and create an axial-symmetric contact surface with the ocular tissue.

23. The system of claim 21, wherein the mechanical property is stiffness.

24. The system of claim 21, wherein the mechanical property is modulus.

25. The system of claim 24, further comprising a diagnoser configured to determine a medical diagnosis based on the modulus.

26. A system, comprising:
means for contacting and indenting an ellipsoidal shell resulting in an indentation region; and
means for determining a mechanical property of the ellipsoidal shell based on indentation data measured from the indentation region.

27. The system of claim 26, further comprising:
means for receiving the indentation data.
